(19) United States
(12) Patent Application Publication
(10) Pub. No.: US 2016/0058275 A1
(43) Pub. Date: Mar. 3, 2016

(54) MANUAL JAW OPENING DEVICE WITH FORCE ACTING ON MOLARS

(71) Applicants: National Taiwan University Hospital, Taipei City (TW); Ming Chi University of Technology, New Taipei City (TW)

(72) Inventors: Chih-Chung HU, New Taipei City (TW); Yi-Chian WANG, Taipei City (TW)

(21) Appl. No.: 14/530,708

(22) Filed: Nov. 1, 2014

(30) Foreign Application Priority Data
Sep. 1, 2014 (TW) 103130119

(51) Int. Cl. A61B 1/24; A61C 19/04

(52) U.S. Cl. CPC A61B 1/24; A61C 19/04

(57) ABSTRACT

A manual jaw opening device with force acting on molars includes: a manual pivotal rotating device; and a pair of opening arms, respectively formed with a first end and a second end, wherein the first end is connected to the manual pivotal rotating device and the second end is equipped with a molar engaging seat; when the pair of opening arms are disposed in the mouth of a patient and the two molar engaging seats are respectively abutted against plural upper molars and plural lower molars, an operator is able to separate the pair of opening arms through operating the manual pivotal rotating device thereby allowing the mouth to be opened.

### BACKGROUND OF THE INVENTION

1. Field of the Invention

The present invention relates to a jaw opening device, especially to a manual jaw opening device with force acting on molars.

2. Description of Related Art

For a patient with his/her mouth opening angle being restrained, the mouth opening practice, mouth cleaning or other mouth-related medical operations may need a jaw opening device for assistance.

A conventional jaw opening device often utilizes the upper and the lower incisors and canines as force acting points, so when a mouth is desired to be opened, the incisors and canines may be subject to a large force. If the operation is not well performed, the incisors or canines are very likely to be damaged.

In addition, the conventional jaw opening device is manually disposed in the frontal portion of the mouth of a patient, so the mouth is able to be opened by the jaw opening device; however, the mouth cleaning or other medical operations may be hard to be carried out by the medical personnel due to the installing location of the jaw opening device.

In view of the above-mentioned disadvantages, a novel jaw opening device shall be provided.

### SUMMARY OF THE INVENTION

One primary objective of the present invention is to provide a jaw opening device, which is capable of preventing the incisors or canines of a patient from being damaged when the mouth of the patient is desired to be opened.

Another objective of the present invention is to provide a jaw opening device, so the medical personnel is able to easily perform mouth opening or other medical operations on a patient when the mouth of the patient is opened.

One another objective of the present invention is to provide a jaw opening device, in which an indication of opening angle is provided as a reference to the operator.

Still one another objective of the present invention is to provide a jaw opening device, wherein through abutting against molars with relatively larger contact areas comparing to other teeth, a sliding movement can be avoided when the jaw opening device is operated.

Still one another objective of the present invention is to provide a jaw opening device, so the syndrome of insufficient mouth opening at single-side or two-side of the month can be treated.

For achieving said objectives, the present invention provides a manual jaw opening device with force acting on molars, which includes:

a manual pivotal rotating device; and

a pair of opening arms, respectively formed with a first end and a second end, wherein the first end is connected to the manual pivotal rotating device and the second end is provided with a molar engaging seat;

wherein, when the pair of opening arms are disposed in the mouth of a patient and the two molar engaging seats are respectively abutted against plural upper molars and plural lower molars, an operator is able to separate the pair of opening arms through operating the manual pivotal rotating device thereby allowing the mouth to be opened.

According to one embodiment provided by the present invention, the manual pivotal rotating device includes two handgrips.

According to one embodiment provided by the present invention, the manual pivotal rotating device further includes two sleeve ends for being connected to the two first ends of the pair of opening arms.

According to one embodiment provided by the present invention, the manual pivotal rotating device further includes an opening angle indication unit.

According to one embodiment provided by the present invention, the molar engaging seat is formed with a rough engaging surface.

According to one embodiment provided by the present invention, the molar engaging seat is formed with plural engaging cavities.

According to one embodiment provided by the present invention, the two molar engaging seats are connected by an elastic member.

According to one embodiment provided by the present invention, the pair of opening arms is made of a metal or an alloy.

According to one embodiment provided by the present invention, the molar engaging seat is made of a metal or an alloy.

According to one embodiment provided by the present invention, a gap is formed between the two molar engaging seats and the pair of opening arms, and the two molar engaging seats are substantially in parallel with the pair of opening arms.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic view illustrating the manual jaw opening device with force acting on molars according to one embodiment of the preset invention;

FIG. 2 is a schematic view illustrating another angle according to the embodiment shown in FIG. 1;

FIG. 3 to FIG. 6 are schematic views illustrating the operating process of the manual jaw opening device with force acting on molars according to the present invention.

FIG. 7 is a schematic view illustrating the manual jaw opening device with force acting on molars according to another embodiment of the preset invention; and

FIG. 8 is a schematic view illustrating the manual jaw opening device with force acting on molars according to one another embodiment of the preset invention.

### DETAILED DESCRIPTION OF THE PREFERRED EMBODIMENT

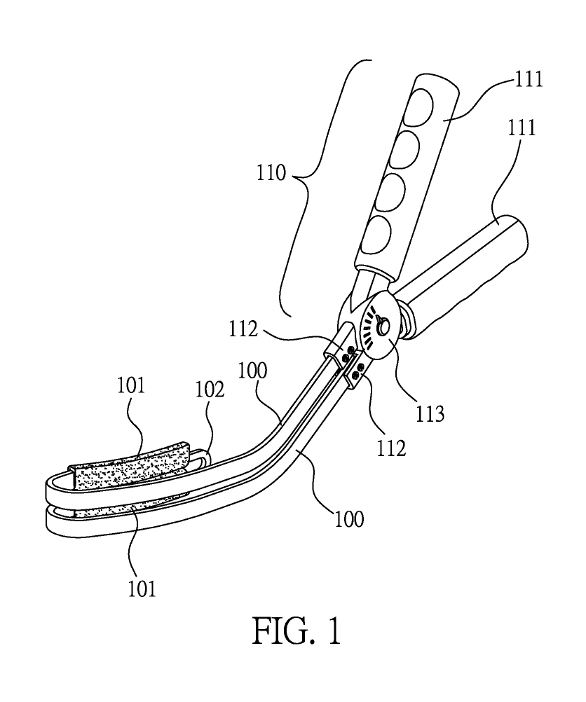

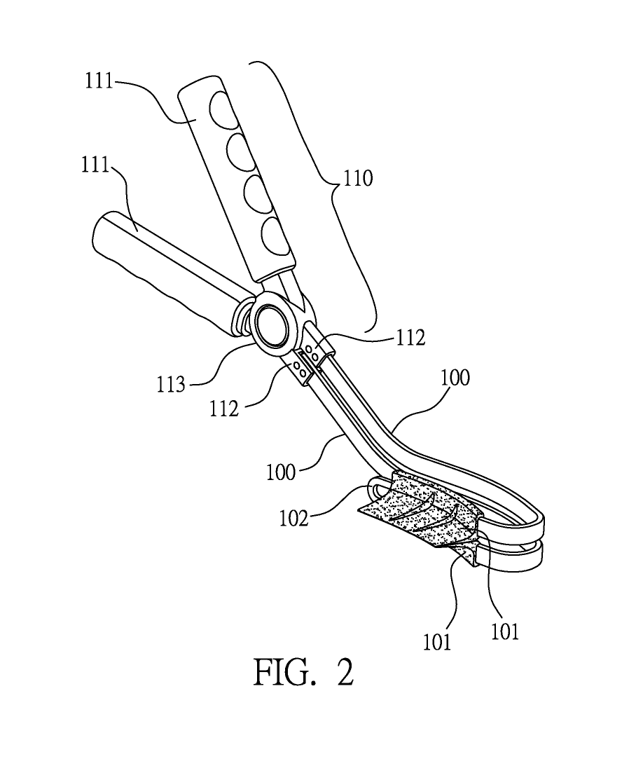

Referring to FIG. 1 and FIG. 2, wherein FIG. 1 is a schematic view illustrating the manual jaw opening device with force acting on molars according to one embodiment of the preset invention; and FIG. 2 is another schematic view illustrating the manual jaw opening device with force acting on molars according to one embodiment of the preset invention. As shown in FIG. 1 and FIG. 2, the present invention provides a manual jaw opening device, which includes a pair of opening arms 100 and a manual pivotal rotating device 110.

The pair of opening arms 100 are respective formed with a first end and a second end, wherein the first end is connected to the manual pivotal rotating device 110, the second end is provided with a molar engaging seat 101, and the two molar engaging seats 101 are connected by an elastic member 102. The pair of opening arms 100 can be made of a metal or an alloy.

The molar engaging seat 101 is formed with a rough engaging surface thereby preventing molars from sliding on the surface of the molar engaging seat 101. The molar engaging seat 101 can be made of a metal or an alloy.

A gap is formed between the two molar engaging seats 101 and the pair of opening arms 100, and the two molar engaging seats 101 are substantially in parallel with the pair of opening arms 100.

The manual pivotal rotating device 110 includes two handgrips 111, two sleeve ends 112 and an opening angle indication unit 113.

The two sleeve ends 112 are connected to the two first ends of the pair of opening arms 100. The connection means for the opening arms 100 and the sleeve ends 112 can be fixedly connected or detachably latched.

The opening angle indication unit 113 is used for indicating the opening angle of the pair of opening arms 100 so as to provide a reference to an operator.

In actual operation, when the pair of opening arms 100 are disposed in the mouth of a patient and the two molar engaging seats 101 are respectively abutted against plural upper molars and plural lower molars, an operator is able to separate the pair of opening arms 100 through operating the manual pivotal rotating device 110 thereby allowing the mouth to be opened.

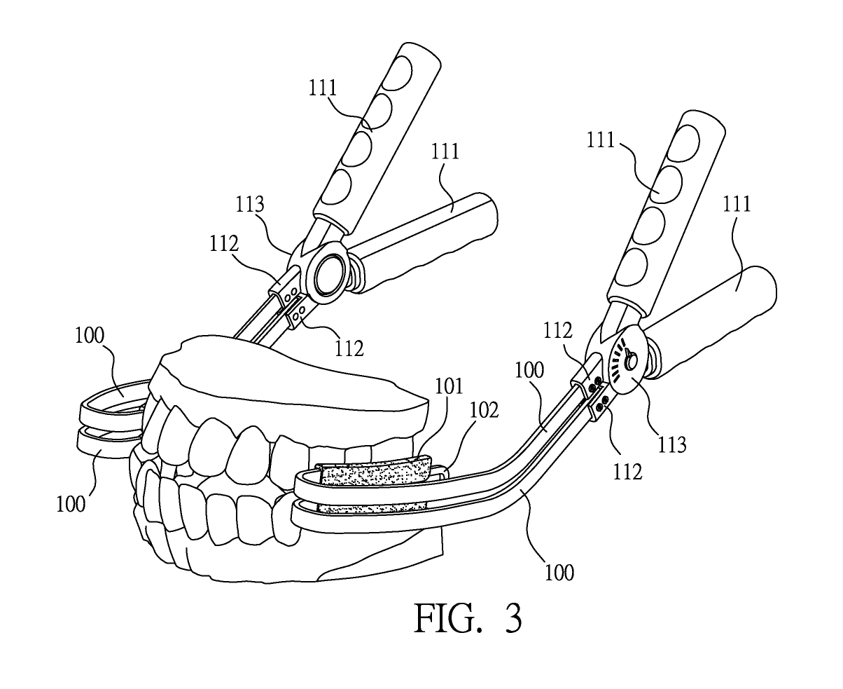

Referring from FIG. 3 to FIG. 6, which are schematic views illustrating the operating process of the manual jaw opening device with force acting on molars according to the present invention.

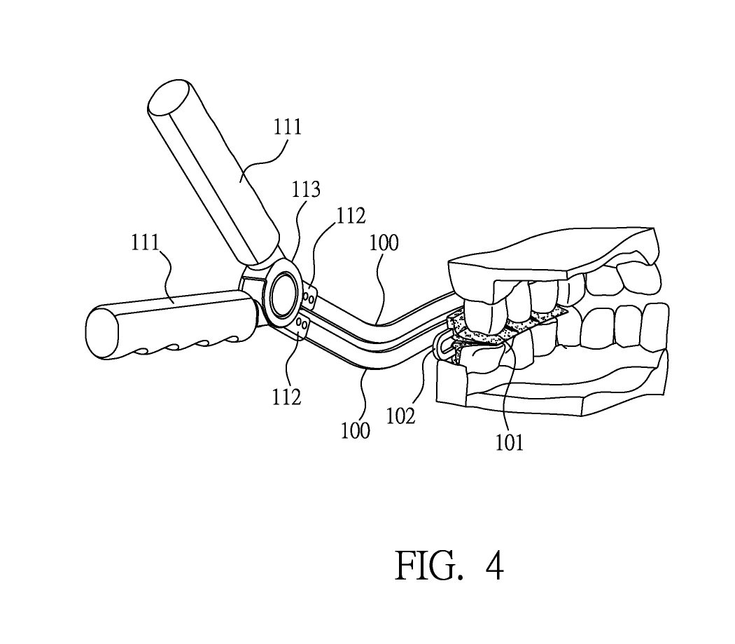

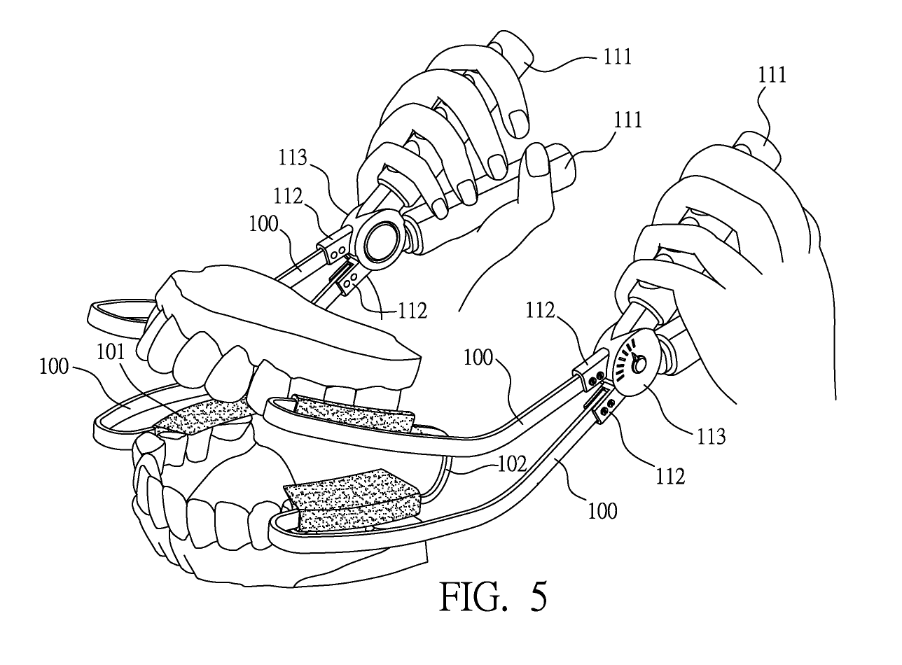

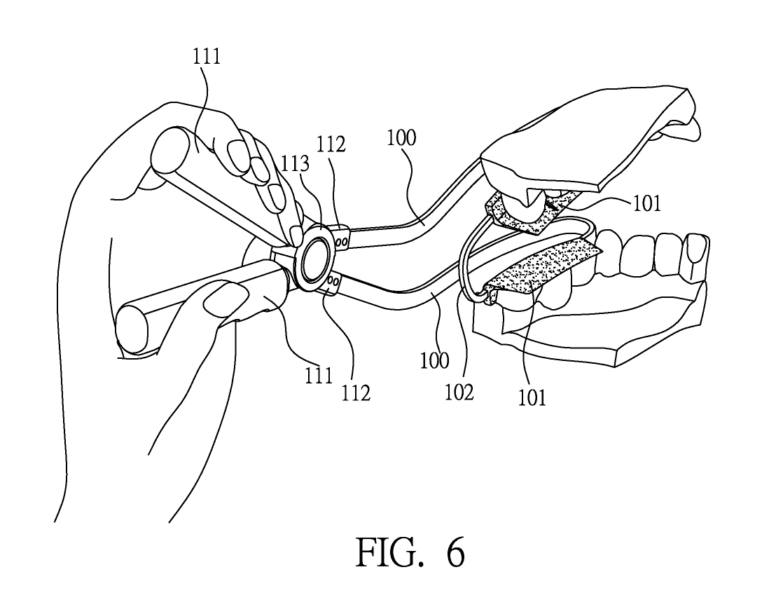

As shown in FIG. 3, in actual operation, firstly an operator respectively dispose two of the above-mentioned manual jaw opening devices with force acting on molars in the left side and the right side of the mouth of a patient, so the two molar engaging seats 101 of each of the manual jaw opening devices with force acting on molars are enabled to be respectively abutted against plural upper molars and plural lower molars, the abutting status is as shown in FIG. 4. As shown in FIG. 5, the operator applies forces on the two handgrips 111 to allow the pair of opening arms 100 to be separated so as to open the mouth. FIG. 6 is a schematic view illustrating the operating process disclosed in FIG. 5 from another angle. When the jaw opening device provided by the present invention is operated on a patient, the above-mentioned manual pivotal rotating device is located at two sides of the cheek and close to the shoulder of the patient, so no interference is generated to the mouth opening or medical operation applied to the patient.

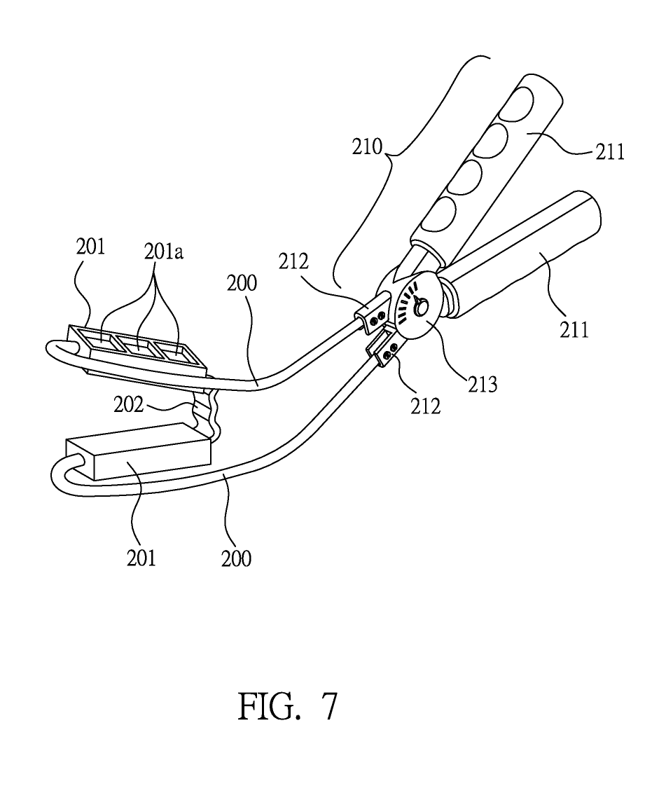

In addition, for preventing the molars from sliding on the engaging surface of the above-mentioned molar engaging seat, the engaging surface is roughened, and the engaging surface can also be formed with plural engaging cavities for receiving the plural molars. Referring to FIG. 7, which is a schematic view illustrating the manual jaw opening device with force acting on molars according to another embodiment of the preset invention. As shown in FIG. 7, the manual jaw opening device includes a pair of opening arms 200 and a manual pivotal rotating device 210.

The pair of opening arms 200 are respective formed with a first end and a second end, wherein the first end is connected to the manual pivotal rotating device 210, the second end is provided with a molar engaging seat 201, and the two molar engaging seats 201 are connected by an elastic member 202. The pair of opening arms 200 can be made of a metal or an alloy.

The molar engaging seat 201 is formed with plural engaging cavities 201a for receiving plural molars, thereby preventing the molars from sliding on the surface of the molar engaging seat 201. The molar engaging seat 201 can be made of a metal or an alloy.

A gap is formed between the two molar engaging seats 201 and the pair of opening arms 200, and the two molar engaging seats 201 are substantially in parallel with the pair of opening arms 200.

The manual pivotal rotating device 210 includes two handgrips 211, two sleeve ends 212 and an opening angle indication unit 213.

The two sleeve ends 212 are connected to the two first ends of the pair of opening arms 200. The connection means for the opening arms 200 and the sleeve ends 212 can be fixedly connected or detachably latched.

The opening angle indication unit 213 is used for indicating the opening angle of the pair of opening arms 200 so as to provide a reference to an operator.

In actual operation, when the pair of opening arms 200 are disposed in the mouth of a patient and the two molar engaging seats 201 are respectively abutted against plural upper molars and plural lower molars, an operator is able to separate the pair of opening arms 200 through operating the manual pivotal rotating device 210 thereby allowing the mouth to be opened.

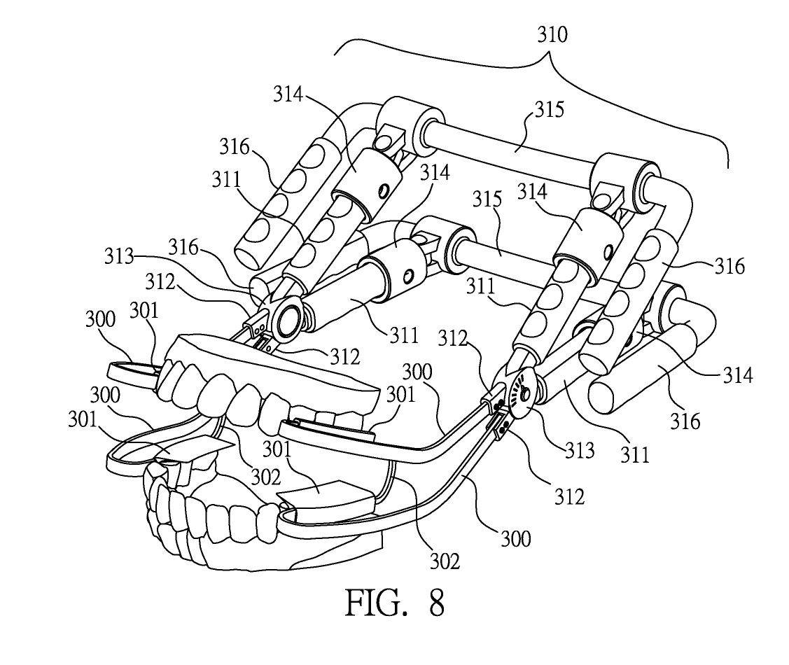

In addition, according to the present invention, a linkage device can be additionally provided for synchronizing two jaw opening devices. Referring to FIG. 8, which is a schematic view illustrating the manual jaw opening device with force acting on molars according to one another embodiment of the preset invention. As shown in FIG. 8, the manual jaw opening device includes two pairs of opening arms 300 and a manual pivotal rotating device 310.

The opening arms 300 are respective formed with a first end and a second end, wherein the first end is connected to the manual pivotal rotating device 310, the second end is provided with a molar engaging seat 301, and the two molar engaging seats 301 are connected by an elastic member 302. The opening arms 300 can be made of a metal or an alloy.

The molar engaging seat 301 is formed with a rough engaging surface thereby preventing molars from sliding on the surface of the molar engaging seat 301. The molar engaging seat 301 can be made of a metal or an alloy.

A gap is formed between the molar engaging seat 301 and the opening arm 300, and the molar engaging seat 301 is substantially in parallel with the opening arm 300.

The manual pivotal rotating device 310 includes two pairs of force arms 311, two pairs of sleeve ends 312, two opening angle indication units 313, two pairs of sleeve units 314, two linkage rods 315 and two pairs of handgrips 316.

Each of the pairs of sleeve ends 312 are connected to the two first ends of the corresponding pair of opening arms 300, the connection means thereof can be fixedly connected or detachably latched.

The opening angle indication unit 313 is used for indicating the opening angle of each of the pairs of opening arms 300 so as to provide a reference to an operator.

The sleeve unit 314 is connected to one end of the force arm 311, and the connection means thereof can be fixedly connected or detachably latched.

Two sides of the linkage rod 315 are respectively provided with one of the sleeve units 314 and one of the handgrips 316.

In actual operation, when the two pairs of opening arms 300 are disposed in the mouth of a patient and the two pairs of molar engaging seats 301 are respectively abutted against plural upper molars and plural lower molars, an operator is able to separate the two pairs of opening arms 300 through operating the manual pivotal rotating device 310 thereby allowing the mouth to be opened.

Based on what has been disclosed above, advantages achieved by the present invention are as following:
1. According to the jaw opening device provided by the present invention, incisors or canines of the patient can be prevented from being damaged when the mouth of the patient is opened.
2. According to the jaw opening device provided by the present invention, the medical personnel is able to easily perform mouth opening or other medical operations on the patient when the mouth of the patient is opened.
3. According to the jaw opening device provided by the present invention, an indication of opening angle is provided as a reference to the operator.
4. According to the jaw opening device provided by the present invention, through abutting against the molars having relatively larger contact areas comparing to other teeth, a sliding movement can be avoided when the jaw opening device is operated.
5. According to the jaw opening device provided by the present invention, the syndrome of insufficient mouth opening at single-side or two-side of the month can be treated.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific examples of the embodiments disclosed and that modifications and other embodiments are intended to be included within the scope of the appended claims. Although specific terms are employed herein, they are used in a generic and descriptive sense only and not for purposes of limitation.

What is claimed is:

1. A manual jaw opening device with force acting on molars, including:
a manual pivotal rotating device; and
a pair of opening arms, respectively formed with a first end and a second end, wherein said first end being connected to said manual pivotal rotating device and said second end being provided with a molar engaging seat;
wherein, when said pair of opening arms being disposed in the mouth of a patient and said two molar engaging seats being respectively abutted against plural upper molars and plural lower molars, an operator being able to separate said pair of opening arms through operating said manual pivotal rotating device thereby allowing said mouth to be opened.

2. The manual jaw opening device with force acting on molars as claimed in claim 1, wherein said manual pivotal rotating device includes two handgrips.

3. The manual jaw opening device with force acting on molars as claimed in claim 2, wherein said manual pivotal rotating device further includes two sleeve ends for being connected to said two first ends of said pair of opening arms.

4. The manual jaw opening device with force acting on molars as claimed in claim 3, wherein said manual pivotal rotating device further includes an opening angle indication unit.

5. The manual jaw opening device with force acting on molars as claimed in claim 1, wherein said molar engaging seat is formed with a rough engaging surface.

6. The manual jaw opening device with force acting on molars as claimed in claim 1, wherein said molar engaging seat is formed with plural engaging cavities.

7. The manual jaw opening device with force acting on molars as claimed in claim 1, wherein said two molar engaging seats are connected by an elastic member.

8. The manual jaw opening device with force acting on molars as claimed in claim 1, wherein said pair of opening arms is made of a metal or an alloy, and said molar engaging seat is made of a metal or an alloy.

9. The manual jaw opening device with force acting on molars as claimed in claim 1, wherein a gap is formed between said two molar engaging seats and said pair of opening arms, and said two molar engaging seats are substantially in parallel with said pair of opening arms.

10. A manual jaw opening device with force acting on molars, including:
a manual pivotal rotating device; and
two pairs of opening arms, each of said pairs of opening arms being respectively formed with a first end and a second end, wherein said first end being connected to said manual pivotal rotating device and said second end being provided with a molar engaging seat;
wherein, when said two pairs of opening arms being disposed in the mouth of a patient and said two pairs of molar engaging seats being respectively abutted against plural upper molars and plural lower molars, an operator being able to separate said pair of opening arms through operating said manual pivotal rotating device thereby allowing said mouth to be opened.
